(19) United States
(12) Patent Application Publication
(10) Pub. No.: US 2025/0195712 A1
(43) Pub. Date: Jun. 19, 2025

(54) AIR PURIFICATION SYSTEMS AND METHODS OF USE THEREOF

(71) Applicant: One Lightwave Manufacturing, Moncton (CA)

(72) Inventors: Derek CUTHBERTSON, Shepody (CA); Eric BLOMSTER, Sarasota, FL (US); Jason O’CONNOR, Shediac Cape (CA)

(73) Assignee: One Lightwave Manufacturing, Moncton, NB (CA)

(21) Appl. No.: 18/538,476

(22) Filed: Dec. 13, 2023

(51) Int. Cl. A61L 9/20

(52) U.S. Cl. CPC A61L 9/20; A61L 2209/134; A61L 2209/14

(57) ABSTRACT

Air purification systems and methods of use thereof are provided. The air purification system may include a housing having an intake, an outlet, and an air flow path extending therebetween. A removable rectangular housing may be positioned within the external housing, an interior of the removable rectangular housing having a reflective surface. In addition, an elongated light may be removeably disposed in removable rectangular housing, such that the elongated light may be actuated to emit light to disinfect an air flow. Accordingly, the system may receive air flow through the intake of the external housing, and direct the air flow across the air flow path through an inlet of the removable rectangular housing, out an outlet of the removable rectangular housing, and out the outlet of the air distribution duct.

### FIELD OF USE

The present disclosure is directed to air purification systems and methods of use thereof, e.g., for neutralizing airborne pathogens.

### BACKGROUND

Indoor air may be two to five times more contaminated than outdoor air. The spread of airborne pathogens such as viruses, bacteria, mold, fungi, pollen, voc's etc. Concern the general population. Air purifiers provide needed health benefits by removing pathogens and particles from the air.

One methods of room decontamination of pathogens is the use of ultraviolet germicidal irradiation (UVGI) equipment. Up to present day a number of short-comings which have plagued the use of UVGI system still have not been adequately been addressed such as all personal, plants, pets, and other organic material must be removed from the room being irradiated to avoid being adversely affected by the UVGI light source. Additional short-comings refer to the shadow effect, distance from the UVGI emitter to the overall area of the room being disinfected, the amount of energy produced, the reflective ability of the surfaces, and the circulation of the volume of air in the room being disinfected.

The use of UVGI energy to kill pathogens poses the issue of shadowing, e.g. when particles are blocked from the UV light by other objects or particles. All particles out of direct line of sight of the UVGI emitter are less effectively disinfected. Some UVGI systems have exposed UV lamps and require the operator to move the device to several locations and run multiple disinfection cycles to complete the process properly. Recently, the newer systems can be either remotely controlled to reposition the unit or can be programmed to reposition at a set location and time. These units are not cost efficient or effective. While in operation, the room cannot be used by personal, all objects that are adversely affected over time by UV-C light waves must be removed from the room before commencement of the disinfection procedure.

Distance is a significant critical consideration due to the rapid drop in the UVGI's ability to decontaminate the further the pathogen is from the emitter. Using some types of systems you must move the equipment to various locations within the room to be effective.

There are a number of critical considerations that have commonly been undervalued in past designs such as the intensity of the UV-C emitter and the duration of exposure time of the pathogen to the emitter, the size of the room, and reflective nature of the walls, does the UVGI lamps used produce wavelengths between 185 and 200 nanometers? If so, then the system is probably generating ozone which can harm the cells in the lungs and respiratory airways.

Another consideration is air circulation within the room. The volume of air in the room must get within the kill zone of the emitter multiple times. The system must be able to draw in air from all parts of the room and expel decontaminated air back into the room.

All UVGI systems that operate using exposed UV-C lamps must have all people, plants, and animals removed from the room during the decontamination process. Exposure can result in damage to eyes and skin even exposed for a few minutes. Multiple short term exposure may have a cumulative effect that may not appear for years.

### SUMMARY

The present disclosure overcomes the drawbacks of previously known systems and methods by providing an air purification system and methods of use thereof. The air purification system may include an external housing having intakes, outlets, and an air flow path extending between the intakes and the outlets. The system further may include one removable rectangular housing positioned within the housing. The one removable rectangular housing may include an inlet and an outlet, such that the air flow path extends between the rectangular housing inlet and the rectangular housing outlet. Moreover, an interior of the removable rectangular housing includes a reflective surface. In addition, the system may include removable elongated lights in the removable rectangular housing, such that the elongated lights may be actuated to emit light, e.g., UVGI light, to disinfect an air flow. Accordingly, the air purification system may receive the air flow through the intake of the rectangular housing, and direct the air flow across the air flow path through the removable rectangular housing inlet and the removable rectangular housing outlet and out the outlet of the external housing. The dimensions of the removable rectangular housing, a reflectivity of the reflective surface, and/or an intensity of the light emitted by the UVGI elongated light may be selected to optimize air flow rate and maximize effectiveness of disinfection of the air flow.

In some embodiments, the removable rectangular housing may include a plurality of elongated lights. Moreover, the interior dimensions of the removable rectangular housing may include a reflective coating having the reflective surface. The system further may include a pre-filter removably coupled to the intake of the external housing, such that the pre-filter may filter particles larger than 3 microns in the air flow at the intakes of the air purification system. In addition, the system may include a fan that may be actuated to cause the air flow to be received through the intakes of the external housing and directed out the outlets of the external housing. Moreover, the system may include a plenum coupled to the outlet of the external housing. The plenum may have outlets in fluid communication with the removable rectangular housing outlet. The system further may include an outlet filter coupled to the outlet of the external housing, such that the outlet filter may filter the air flow at the outlet of the external housing. For example, the outlet filter may be a carbon filter, e.g., having at least one of an activated carbon, a potassium permanganate, or an activated alumina. The system may include an outlet filter, e.g. electrostatic, coupled to the outlet of the external housing located between the outlet filter, e.g. activated carbon filter and the plenum of the external housing. The system further may include air distribution ducts coupled to the outlets of the plenum which may contain air outlets ports directing air flow in a downward direction and directed back towards the system air intakes by means of louvered nozzles within the air outlet ports.

In accordance with another aspect of the present invention, a method for purifying air is provided. The method may include receiving, through the intake of the external housing, the air flow; directing the air flow through the inlet of the removable rectangular housing in fluid communication with the intakes of the external housing; emitting light via the elongated lights disposed within the removable rectangular housing to disinfect the air flow; and directing the disinfected air flow through the outlet of the removable rectangular housing and out the outlet of the plenum. The method further may include filtering the air flow at the intake via the removable prefilter coupled to the intake of the external housing. In addition, the method may include actuating the removable fan coupled to the external housing to cause the air flow to be received through the intake of the housing. Moreover, the method may include filtering the air flow at the outlet of the external housing via the outlet filters coupled to the outlet of the external housing.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1. illustrates an exemplary air purification system constructed in accordance with the principles of the present disclosure.

FIG. 2. is a view of the air purification system without the air distribution ducts.

FIG. 3. is an interior view of the air purification system.

FIG. 4. is an interior view of the interior electrical door panel.

FIG. 5. is a view of the air purification system with the removable rectangular housing component containing UVGI lamps separated from the main body.

FIG. 6. is a view of the air purification system with the removable rectangular housing component separated from the main body with a view of a row of UVGI lamps.

FIG. 7. is a view of the air purification system with the removable motor component separated from the main body.

FIG. 8. is a view of the air purification system with the three removable filter components separated from the main body.

FIG. 9. is a view of a UVGI lamp and titanium quartz tube.

FIG. 10. is a sectional view of the air distribution duct with an air outlet port.

FIG. 11. is a view of the louvered nozzle.

### DETAILED DESCRIPTION

The disclosure will be described more fully hereinafter with reference to the accompanying drawings, in which example embodiments of the disclosure are shown. This disclosure may, however, be embodied in many different forms and should not be construed as limited to the example embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described example embodiments but should be defined only in accordance with the following claims and their equivalents. The description below has been presented for the purposes of illustration and is not intended to be exhaustive or to be limited to the precise form disclosed. It should be understood that alternate implementations may be used in any combination to form additional hybrid implementations of the present disclosure. For example, any of the functionality described with respect to a particular device/component may be performed by another device/component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments.

Certain words and phrases are used herein solely for convenience and such words and terms should be interpreted as referring to various objects and actions that are generally understood in various forms and equivalencies by persons of ordinary skill in the art.

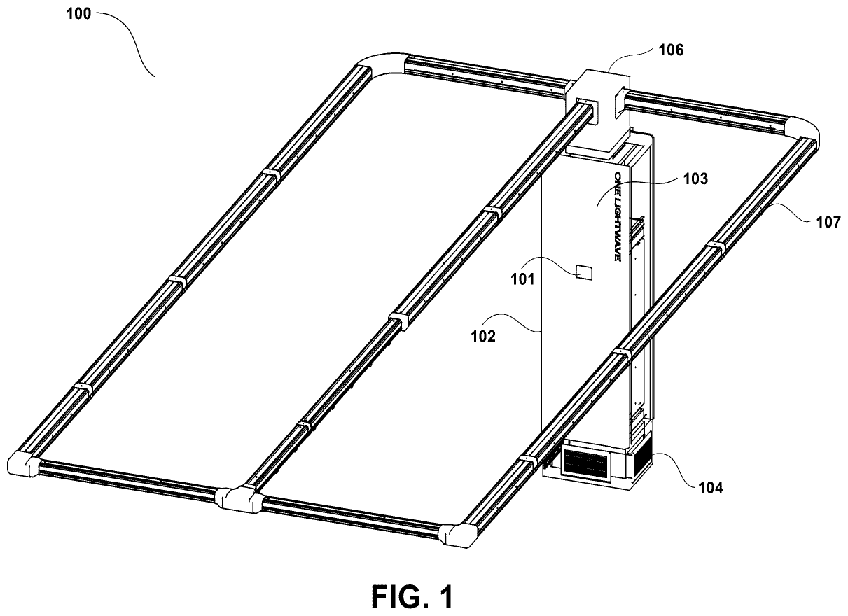

Referring now to FIGS. 1, air purification system 100 is provided. FIG. 1 is a perspective view of a front side of system 100. As shown in FIG. 1, system 100 may include a external housing having body 102, control panel 101, access door 103, and plenum 106. For example, access door 103 may be pivotally coupled to body 102 such that access door 103 may transition between a first closed configuration where access door 103 is engaged with body 102 and a second open configuration where access door 103 is disengaged with body 102, thereby exposing the interior of body 102. Intake door 103 may be sealed when engaged with body 102 such that no air is permitted to escape system 100 except through vents 108, as described in further detail below. Moreover, access door 103 may include lock 105 for locking intake door 103 to body 102 when access door 103 is in its first configuration, to prevent inadvertent opening of access door 103.

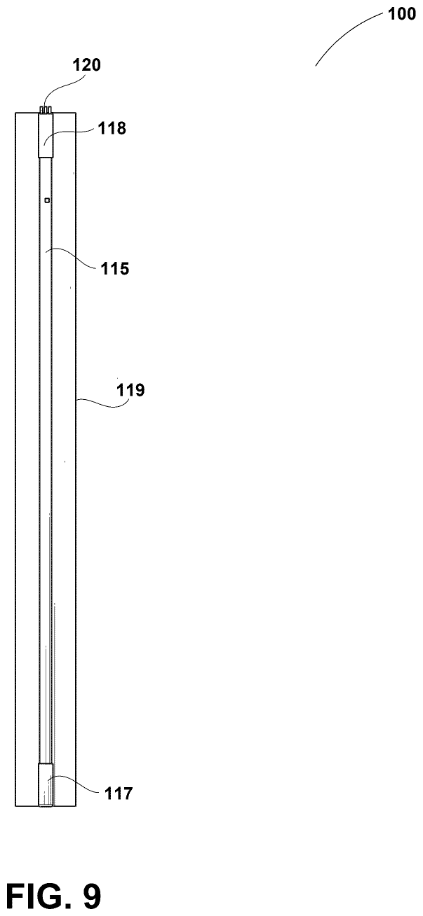

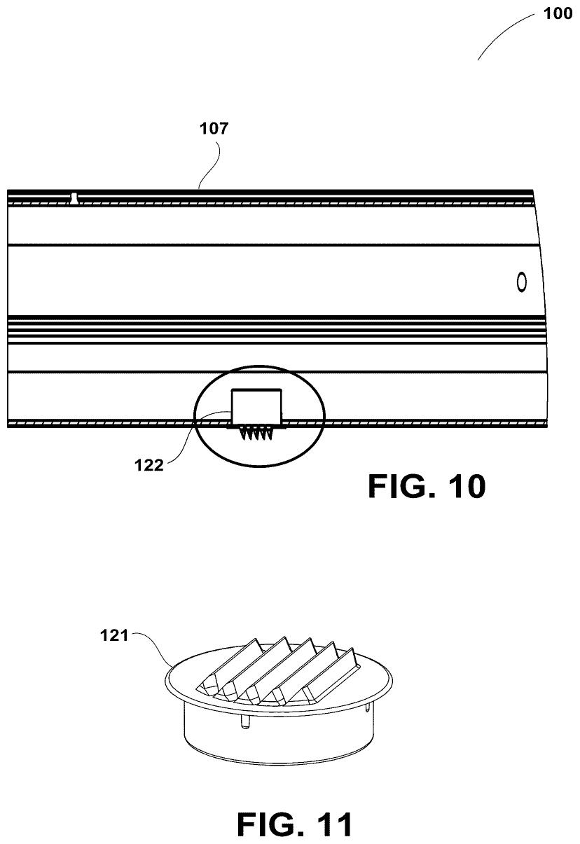

As shown in FIG. 1, at the bottom of system 100, at the front and both sides may include vents 104 to permit air to flow therethrough into the interior of body 102. Moreover, system 100 may include pre-filter 109 disposed adjacent to intake door 103, e.g., within body 102 such that air may flow through vents 104 and across pre-filter 109 before entering the interior of body 102 to thereby filter the air flow upon entry into system 100. Pre-filter 109 may be removable such that it may be replaced or cleaned, as shown in FIG. 9. For example, body 102 may include rail mechanism 123 sized and shaped to removably receive and house filter 109.

As shown in FIG. 1, at the front and both sides of the plenum 106 may be vents 108 to permit air to flow therethrough from the interior of plenum 106 to duct 107 to outside system 100 as shown in FIG. 1.

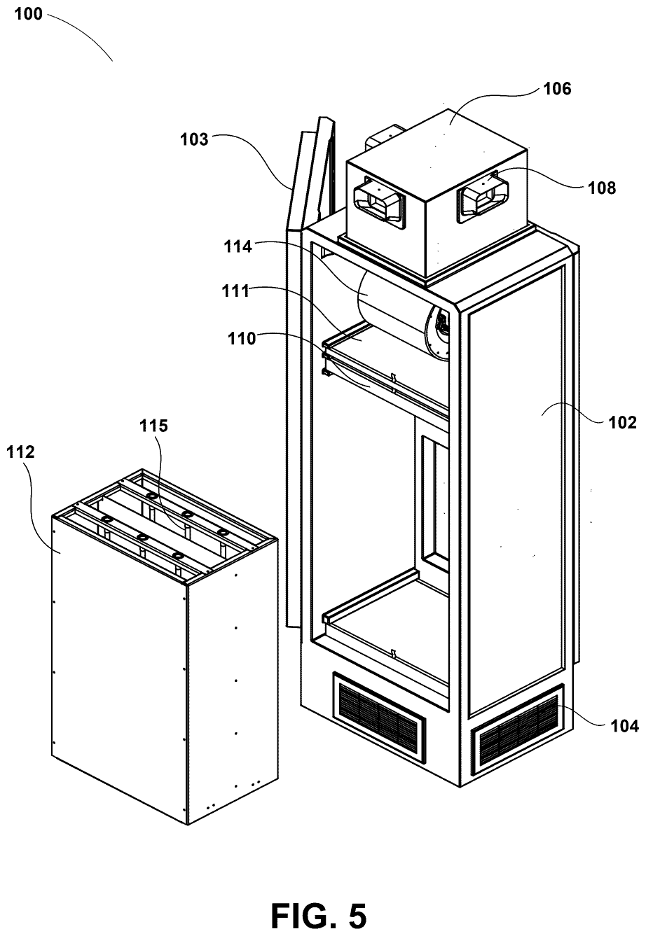

Moreover, system 100 may include filter 110 disposed on top of removable rectangular housing 112, such that air may flow across filter 110 and filter 111 disposed on top of filter 110, such that air may flow across filter 111 through vents 108 before leaving the interior of plenum 106 to thereby filter the air flow upon exit of system 100. For example, filter 110 may filter for odour and volatile organic compounds. Filter 110 may be removable such that it may be replaced or cleaned. In addition, filter 110 may be a carbon filter. For example, the carbon filter may include at least one of an activated carbon, a 15 potassium permanganate, or an activated alumina. For example, filter 111 may filter for small decontaminated compounds. Filter 111 may be removable such that it may be replaced or cleaned. In addition, filter 111 may be an electrostatic filter as shown in FIG. 5.

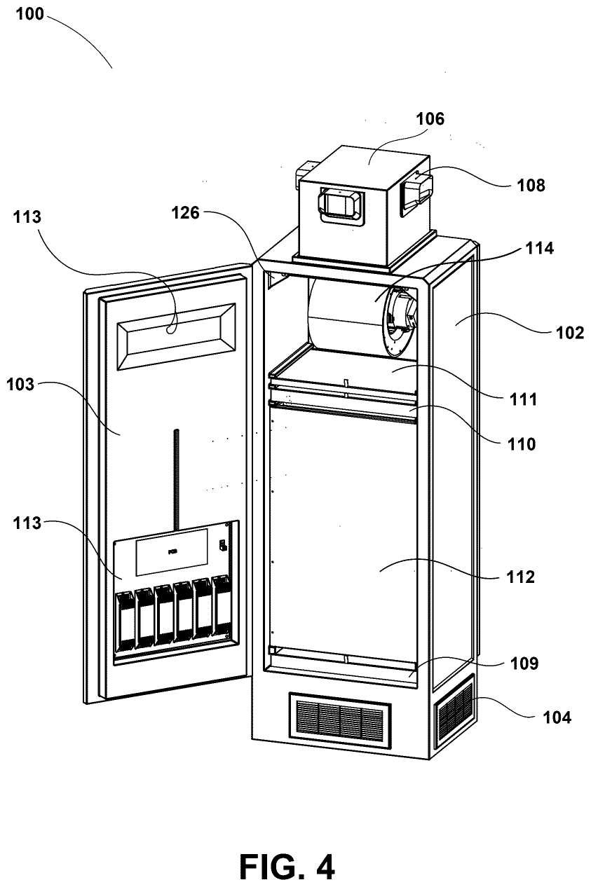

Front door 103 may be pivotally coupled to body 102 such that front door 103 is disengaged with body 102, thereby exposing the interior of a compartment of body 102, e.g., the compartment where a control panel and electrical components 113 of system 100 are stored, as shown in FIG. 4. Electrical components 113 may include a power supply for powering a fan to generate an air flow through system 100 as well as elongated lights to disinfect the air flow, as described in further detail below.

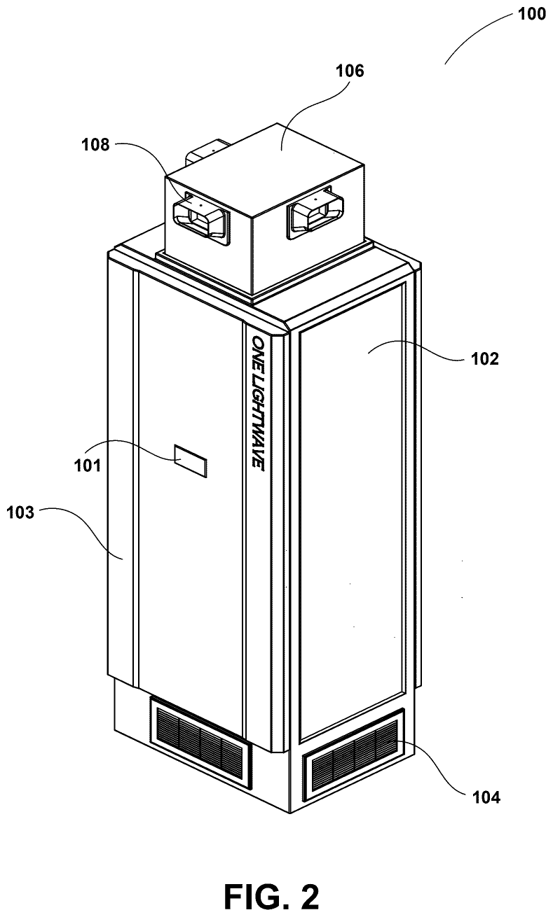

Referring to FIG. 2, door 103 may include vent for providing air flow in and out of the compartment of body 102 storing the electrical components to thereby prevent overheating of the electrical components. Moreover, the airflow drawn in through vent may flow past the electrical components and through additional vents (not shown) located below the electrical components, and into passageway within body 102.

Referring now to FIG. 4, an interior of system 100 is provided. As shown in FIG. 4, system 100 may include removable rectangular housing 112 disposed within body 102. Upper of removable rectangular housing 112 may include an opening to receive plurality of elongated lamps 115.

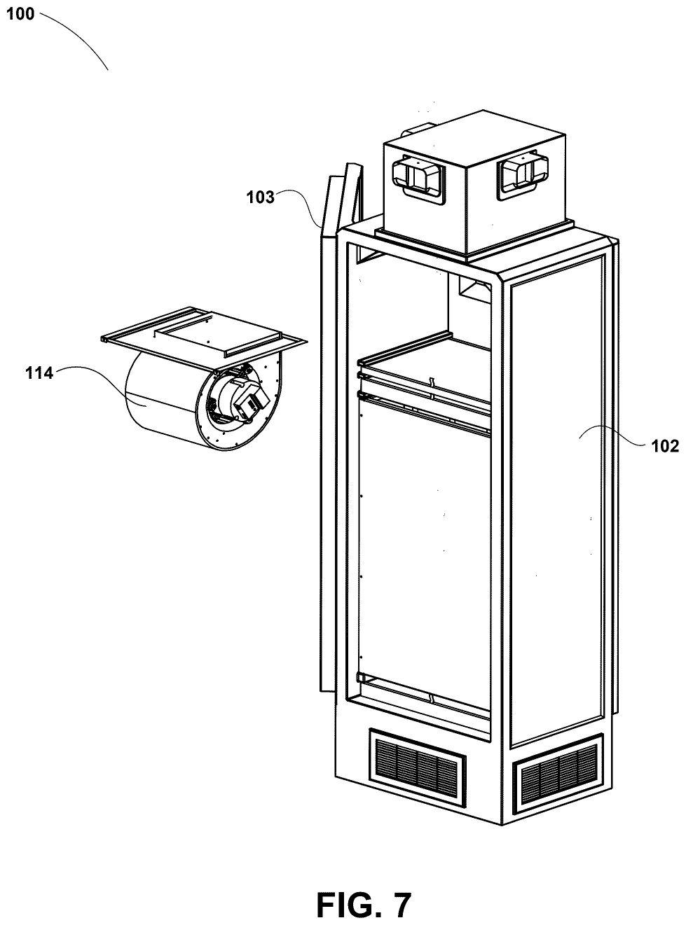

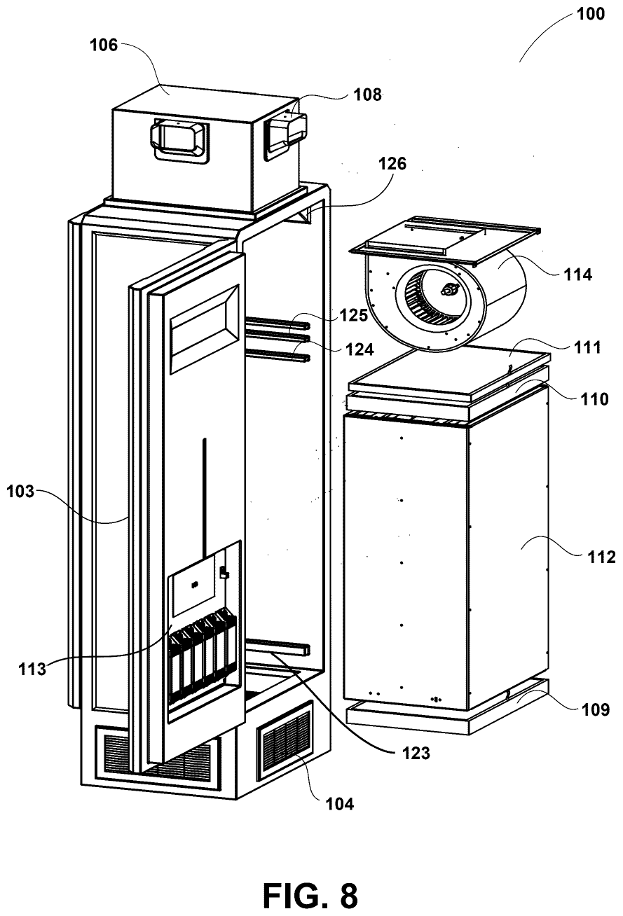

As shown in FIG. 7, system 100 further may include fan motor 114 and may include a rail mechanism 126 sized and shaped to removably receive and house fan motor operatively coupled to top interior of the external housing 102. Plenum 106 may extend from fan motor 114 positioned on top of external housing 102.

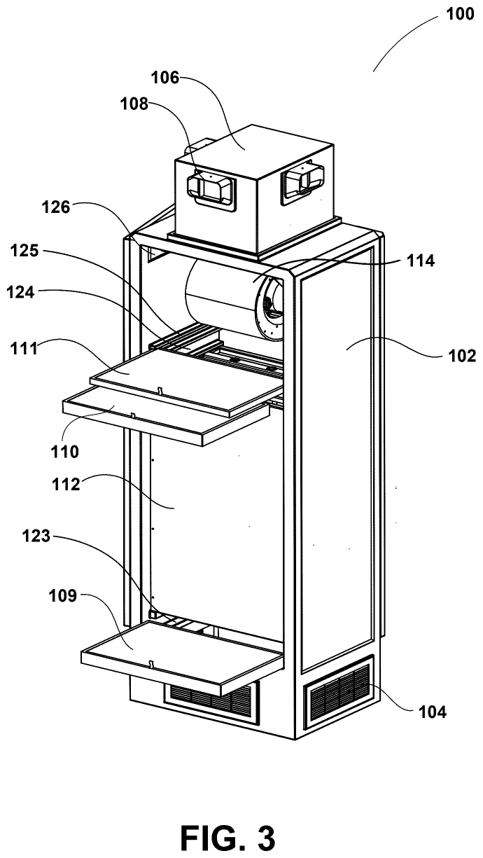

Referring again to FIG. 3, door 103 may include compartment for storing a control panel 101 and electrical components 113 as described above. Electrical components 113 may be electrically coupled to the components of system 100, e.g., fan motor 114 and the plurality of elongated lights as described in further detail below. Accordingly, fan motor 114 may be actuated via electrical components 113 to generate an air flow through system 100. For example, the actuation of fan motor 114 may create a negative pressure vacuum within the interior of the housing of system 100, and cause air to flow through vents 104 of lower external housing 102, across pre-filter 109 and into removable rectangular housing 112. The air flow is then directed through removable rectangular housing of elongated ultraviolet lamps, through the passageway, through filter 110, through filter 111 across fan motor 114, and exits system 100 via outlets 108 of plenum 106.

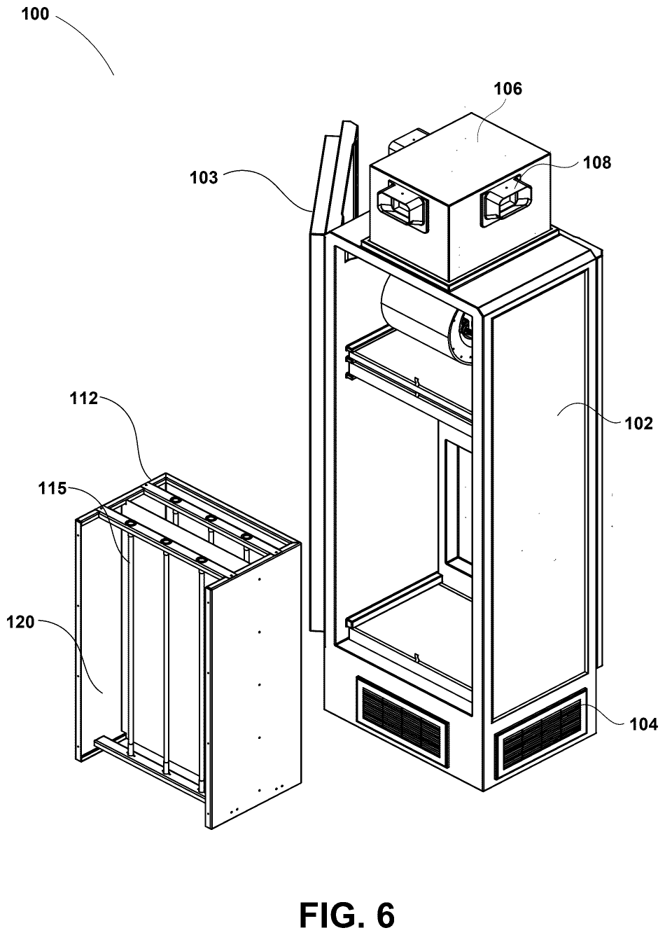

Referring now to FIG. 6, interior is illustrated with front plate removed. As shown in FIG. 6, system 100 may include six elongated lights. As will be understood by a person having ordinary skill in the art, system 100 may include less than six elongated lights, e.g., one, two, three, four, five, or six elongated lights, or alternatively, may include more than six elongated lights. As shown in FIG. 6, each plurality of elongated lights 115 may be actuated to emit light, e.g., UVGI light, to disinfect the air flow through the passageway of removable rectangular housing.

For example, the UVGI light may be absorbed by RNA and DNA in cells and microbes which induces changes in the DNA and RNA structures that result in their inability to replicate. Many microbes have proved to be susceptible to inactivation using UVGI light including bacteria, viruses, fungi, and spores. The amount of inactivation is directly proportional to the UVGI dose received as a result of its intensity, the duration of exposure, and the type of pathogen being exposed.

The farther away the light source, the less UVGI will reach the target, so only a quarter of the UVGI remains when the distance doubles. UVGI radiation has a short wavelength and high energy, which enables for it to function the best in a direct line of sight at a short distance. The UVGI light emitted from elongated lights 115 is able to go into the pores of reflective material 116, which determines reflectivity and intensity of the power output of the lights 115.

As shown in FIG. 4, elongated light 115 may include ceramic ends 118, 117, which sandwich the transparent filament of elongated light 115. Accordingly, the overall length of elongated light 115 including ends 118, 117 may be substantially equal to the length of elongated tube 119. Thus, elongated light 115 may be disposed completely within elongated tube 119.

Alternatively, elongated light 115 may have a length that is shorter than elongated tube 119, thereby reducing the amount of light that travels out of elongated tube 119 and into interior 112. Moreover, elongated light 115 may be slidably removable within a tubing, e.g., made of titanium quartz, within elongated tube 119 such that elongated light 115 may be pulled out, cleaned, and reinserted.

Moreover, the inner diameter of elongated tube 119 may have a reflective surface, such that light emitted from elongated light 115 may be reflected via the circular reflective surface. Accordingly, disinfection of the air flow through elongated tube 119 will be maximized as no particles within the air flow will be blocked or “shadowed” by another particle within the air flow. For example, the emitted light waves will bounce any which way within elongated tube 119, to thereby attack particles at every angle. In some embodiments, elongated tube 119 may be formed of a reflective material. Additionally or alternatively, the inner surface of elongated tube 119 may include a reflective coating. In addition, elongated light 115 may include electrical coupler 120, e.g., disposed on ceramic end 118, for electrically coupling elongated light 115 with electrical components 113 of system 100.

The inner diameter of elongated tubes 119, the reflectivity of the reflective surface of elongated tubes 119, and/or the intensity of the light emitted by elongated lights 115 may be selected to optimize air flow rate and maximize effectiveness of disinfection of the air flow. In one embodiment, for example, elongated tubes 119 may have a length of 39 inches, and accordingly elongated lights 115 may have an overall length of 39 inches such that ceramic ends 118, 117 each have a length of 1.5 inches, and the strand of filament has a length of 36 inches. Moreover, elongated tubes 119 may have a diameter such that no particle within the air flow passing through removable rectangular housing 112 will be further than *** inches from the light source, e.g., elongated lights 115. For example, with a strength of 20,000 mW/cm², it would take roughly 0.5 seconds to effectively irradiate influenza in the air passing through removable rectangular housing 112. However, when the energy is quadrupled to 85,000 mW/cm², it may only take ⅛th of a second to irradiate influenza in the air passing through removable rectangular housing 112. As will be understood by a person having ordinary skill in the art, the inner diameter of removable rectangular housing 112, the reflectivity of the reflective surface of removable rectangular housing 112, and the intensity of the light emitted by elongated lights 115 may proportionally be scaled up or down for various applications.

FIG. 3 is a view of the components. Specifically, FIG. 3 illustrates vents 104, filter 109, pre-filter rail 123, volatile organic compound (voc) filter 110, VOC filter rail 124, electrostatic filter 111, electrostatic filter rail 125, fan motor 114, fan motor rail 126, and outlet port 108. Outlet port 108 may include a plurality of ports to permit air to flow therethrough from outlet plenum 106 to air distribution duct 107 through the outlet port 108.

Referring now to FIG. 4, exemplary method 600 for using air purification system 100 is provided. At step 601, fan motor 114 may be actuated to create a negative pressure vacuum within external housing 102, thereby causing air flow to enter system 100 through vents 104 into passageway 120 of body 102. As described above, pre-filter 109 may filter the air flow at the intake. At step 604, the air flow is directed through passageway 120 and into removable rectangular housing 112. At step 606, light, e.g., UVGI light, is emitted from elongated lights 115 positioned within removable rectangular housing 112. For example, the UVGI light reflects off of the reflective surface and/or coating of the inner surface of removable rectangular housing 112 to thereby attack particles within the air flow at every angle. At step 608, the disinfected air flow is directed out of removable rectangular housing 12, flows across fan motor 114 and enters plenum 106, and exits outlet ports 108 of plenum to air distribution ducts 107 and exits system 100 via louvered nozzle 121. As described above, filter 110, and filter 111 may filter the disinfected air flow before it exits system 100, e.g., for odour and volatile organic compounds and particles.

The systems and methods disclosed herein may be incorporated into industrial air purification systems, residential air purification systems, portable air purification systems, nonportable air purification systems, HVAC systems, counter top systems, or the like. That is, the systems and methods described herein may be scaled up, scaled down, or reconfigured depending on the space (rooms/building) in which the air is being filtered and/or purified.

While various illustrative embodiments of the invention are described above, it will be apparent to one skilled in the art that various changes and modifications may be made therein without departing from the invention. The appended claims are intended to cover all such changes and modifications that fall within the true scope of the invention.

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

### ITEMS

101 Control Panel
102 External Housing
103 Door
104 Intake Vent
105 Door Lock
106 Plenum
107 Air Distribution Duct
108 Outlet Ports
109 Pre-filter
110 VOC Filter
111 Electrostatic Filter
112 Removable Rectangular Housing
113 Electrical Components
114 Fan Motor
115 UVGI Lamp
116 Reflective Material
117 Ceramic Ends
118 Ceramic Ends
119 Titanium Quartz Tube
120 Passage Way
121 Louvered Nozzle
122 Air outlet Port
123 Rail for Pre-filter
124 Rail for VOC Filter
125 Rail for Electrostatic Filter
126 Rail for Fan Motor

1. An air purification system comprising:
a housing comprising an intake, an outlet, and an air flow path extending between the intake and the outlet;
at least one removable rectangular housing positioned within the external housing, such that the air flow path extends between the removable rectangular housing inlet and the removable rectangular housing outlet, an interior comprising a reflective surface; and
an elongated light removeably disposed in the removable rectangular housing, the elongated light configured to emit light to disinfect an air flow, wherein the system is configured to receive the air flow through the intake of the external housing, and direct the air flow across the air flow path through the removable rectangular housing inlet and the removable rectangular housing outlet and out the outlet of the external housing.

2. The system of claim 1, wherein the removable rectangular housing comprises a plurality of elongated lights.

3. The system of claim 1, wherein the interior of the removable rectangular housing comprises a reflective coating comprising the reflective surface such that the reflective coating magnifies the intensity of the elongated light.

4. The system of claim 1, wherein the elongated light is configured to emit UVGI light to disinfect the air flow.

5. The system of claim 1, wherein an inner dimension of the removable rectangular housing, a reflectivity of the reflective surface, and an intensity of the light emitted by the elongated light are selected to optimize air flow rate and maximize effectiveness of disinfection of the air flow.

6. The system of claim 1, further comprising a pre-filter removably coupled to the intake of the housing, the pre-filter configured to filter the air flow at the intake.

7. The system of claim 1, further comprising a fan motor configured to cause the air flow to be received through the intake of the housing and directed out the outlet of the housing.

8. The system of claim 1, further comprising a plenum coupled to the outlet of the external housing, the plenum having a plenum inlet in fluid communication with the removable rectangular housing outlet of the removable rectangular housing.

9. The system of claim 8, wherein the plenum inlet extends beyond the removable rectangular housing outlet of the removable rectangular housing.

10. The system of claim 1, further comprising two outlet filters coupled to the outlet of the external housing, the outlet filters configured to filter the air flow at the outlet of the external housing.

11. The system of claim 10, wherein the first outlet filter comprises a carbon filter and the second outlet filter comprises an electrostatic filter.

12. The system of claim 11, wherein the carbon filter comprises at least one of an activated carbon, a potassium permanganate, or an activated alumina.

13. The system of claim 9, further comprising three outlet ports in fluid communication with removable air distribution duct.

14. The system of claim 13, further comprising an air distribution duct extending from each of three outlet ports, the two air distribution ducts coupled to the side outlet ports conversing the length of the two area walls, an air distribution duct connects the two ducts, the third air distribution duct is coupled to the center outlet port conversing the center length of the area, connected to the duct joining the aforementioned ducts.

15. The system of claim 14 comprising evenly spaced louvered nozzles directing air flow downwards and back towards the housing.
